(19) United States
(12) Patent Application Publication
(10) Pub. No.: US 2006/0153636 A1
(43) Pub. Date: Jul. 13, 2006

(54) Elbow structure of a musical instrument stand

(76) Inventor: Ming Yi Chang, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/030,874

(22) Filed: Jan. 10, 2005

(51) Int. Cl. A61B 17/00

(52) U.S. Cl. 403/400

(57) ABSTRACT

An elbow structure of a musical instrument stand. The elbow structure includes an elbow formed with a transverse circular hole and a rotary block rotatably fitted in the circular hole. The elbow is further formed with a cut intersecting the circular hole. Two sides of the cut are respectively formed with two arced rails. The rotary block is formed with a radially extending cavity. A top wall of the cavity is formed with a fixing member through hole. The rotary block is further formed with a through section communicating with the cavity. A fixing member having a ring section is inlaid in the cavity. One end of an adjustment rod is fitted through the through section of the rotary block. The ring section of the fixing member is formed with a through hole for the adjustment rod to pass through. A threaded pillar extending from outer circumference of the ring section is fitted through the fixing member through hole of the cavity. The ring section is positioned in the cavity. An outer end of the threaded pillar extends through a pad member bridged between the arced rails. A rotary member is screwed on the threaded pillar to press the pad member against the arced rails.

### BACKGROUND OF THE INVENTION

The present invention is related to an elbow structure of a musical instrument stand, and more particularly to an elbow structure for supporting a percussion instrument. The elbow structure includes an elbow, a rotary block, a fixing member and a rotary member for fixing an adjustment rod. The angle and the extending length of the adjustment rod can be adjusted at one time.

FIGS. 9 and 10 show a musical instrument stand for percussion instruments such as jazz drum and cymbal. The cymbal 9 is supported by an inclined rod 91. The inclined rod 91 is fixedly connected on a stand 92 via an elbow 8. The elbow 8 includes a seat body 81 having a circular coupling seat 82 on upper side for coupling with a circular rotary block 83. The rotary block 83 is formed with an axial thread hole 831 and a radial through hole 832. The inclined rod 91 extends through the through hole 832. A first bolt 84 is inserted through the coupling seat 82 and screwed into the thread hole 831 of the rotary block for fixing the rotary block 83. The coupling faces of the coupling seat 82 and the rotary block 83 are formed with cooperative toothed sections 821, 833 for preventing the rotary block from rotating. A second bolt 85 is screwed into one end of the thread hole 831 of the rotary block opposite to the first bolt 84 for fixing the inclined rod 91.

In use, the first bolt is unscrewed and loosened, whereby the rotary block can be rotated to adjust the inclination angle of the inclined rod. Then the second bolt can be unscrewed and loosened to adjust the length of the inclined rod. Accordingly, the cymbal can be adjusted to an optimal position. Then the first and second bolts are tightened to fix the cymbal in the optimal position.

The end face of the second bolt directly abuts against the inclined rod to tighten and fix the inclined rod. The contact area between the inclined rod and the second bolt is simply the area of the end face of the second bolt. Therefore, under striking force, the inclined rod tends to loosen and slip down. As a result, the cymbal is easy to displace from the optimal position. Therefore, it is necessary for a performer to again adjust the position of the cymbal.

Furthermore, when adjusting the height and inclination angle of the inclined rod, it is necessary to respectively rotate the first and second bolts. This is quite inconvenient. In addition, the adjustment of angle is limited by the pitch between the teeth of the toothed sections. Therefore, the cymbal cannot be micro-adjusted.

### SUMMARY OF THE INVENTION

It is therefore a primary object of the present invention to provide an elbow structure of a musical instrument stand. The elbow structure includes an elbow and a fixing member fitted on an adjustment rod. When the fixing member is tightened, the adjustment rod is tightly fastened on the elbow. The fixing member is inlaid in a rotary block disposed in the elbow. When the fixing member is loosened, the angle and the extending length of the adjustment rod can be adjusted at one time.

According to the above object, the elbow structure of the musical instrument stand of the present invention includes an elbow formed with a transverse circular hole and a rotary block rotatably fitted in the circular hole. The elbow is further formed with a cut intersecting the circular hole. Two sides of the cut are respectively formed with two arced rails. The rotary block is formed with a radially extending cavity. A top wall of the cavity is formed with a fixing member through hole. The rotary block is further formed with a through section communicating with the cavity. A fixing member having a ring section is inlaid in the cavity. One end of an adjustment rod is fitted through the through section of the rotary block. The ring section of the fixing member is formed with a through hole for the adjustment rod to pass through. A threaded pillar extending from outer circumference of the ring section is fitted through the fixing member through hole of the cavity. The ring section is positioned in the cavity. An outer end of the threaded pillar extends through a pad member bridged between the arced rails. A rotary member is screwed on the threaded pillar to press the pad member against the arced rails.

The present invention can be best understood through the following description and accompanying drawings wherein:

### BRIEF DESCRIPTION OF THE DRAWINGS

FIG. 1 is a perspective assembled view of a first embodiment of the elbow structure of the present invention;

FIG. 2 is a perspective exploded view of the first embodiment of the elbow structure of the present invention;

FIG. 3 is a sectional assembled view of the first embodiment of the elbow structure of the present invention;

FIG. 4 is another sectional assembled view of the first embodiment of the elbow structure of the present invention;

FIG. 5 is a perspective partially sectional view of the rotary block of a second embodiment of the present invention;

FIG. 6 is a perspective partially sectional view of the rotary block of a third embodiment of the present invention;

FIG. 7 is a perspective partially sectional view of the rotary block of a fourth embodiment of the present invention;

FIG. 8 is a plane assembled view of a fifth embodiment of the elbow structure of the present invention;

FIG. 9 is a perspective view of a conventional musical instrument stand for supporting a cymbal; and

FIG. 10 is a perspective exploded view of the elbow structure of the conventional musical instrument stand.

### DETAILED DESCRIPTION OF THE PREFERRED EMBODIMENTS

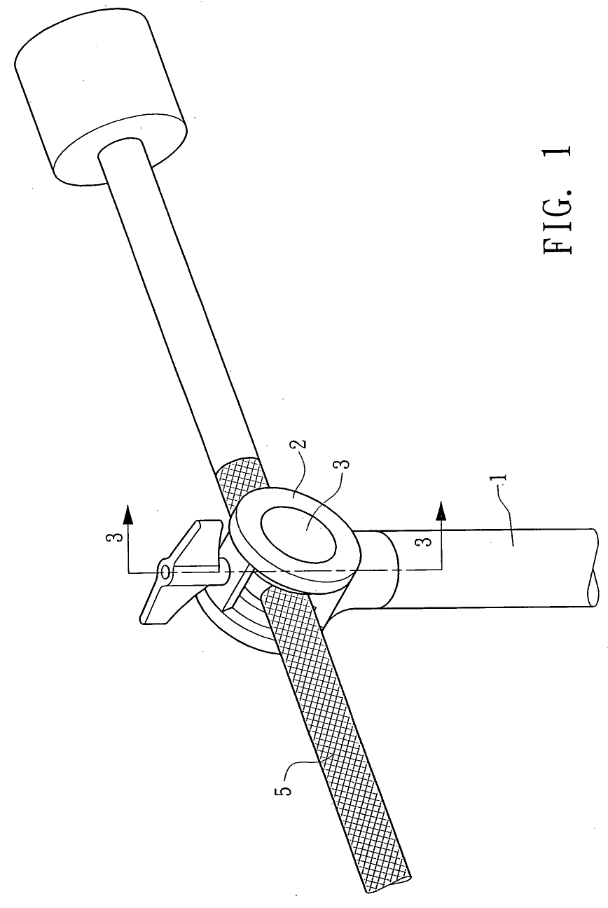

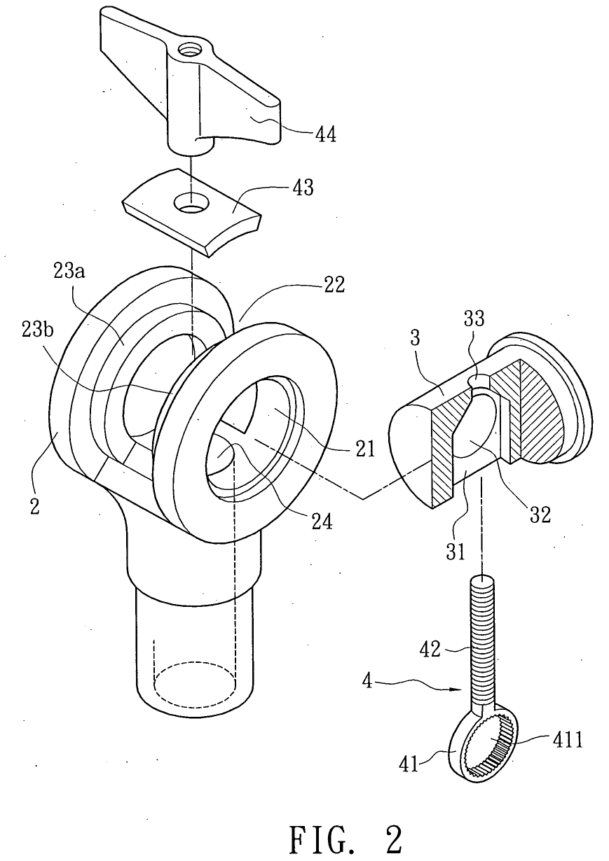

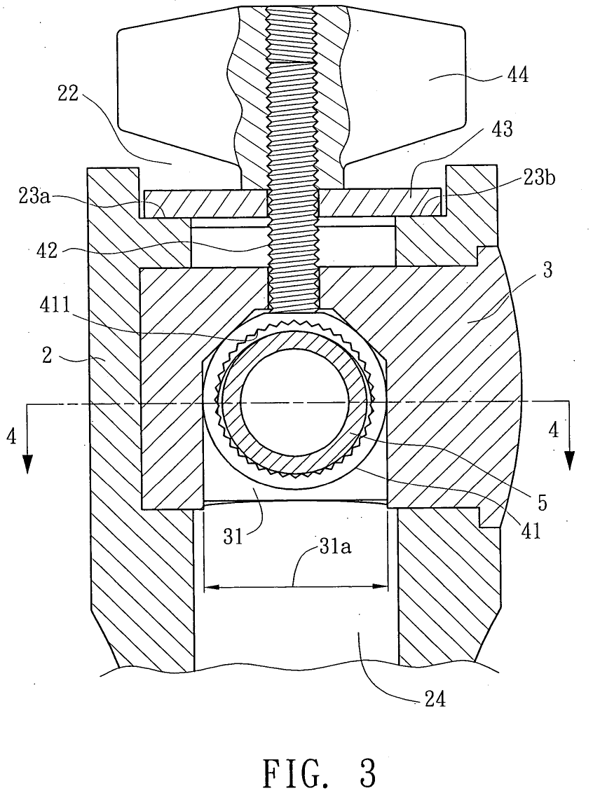

Please refer to FIGS. 1 to 3. According to a first embodiment, the elbow 2 of the musical instrument stand of the present invention is connected with an upper end of an upright stem 1 of the stand. The elbow 2 is formed with a transverse circular hole 21 through which a rotary block 3 is fitted. The elbow 2 is also formed with a cut 22 intersecting the circular hole 21. The cut 22 radially extends within a predetermined arc range of the circular hole 21. Two sides of the cut 22 are respectively formed with two arced rails 23a, 23b concentric with the circular hole 21. The bottom of the cut 22 is formed with a stem hole 24 axially communicating with the interior of the upright stem 1 connected with the elbow.

In this embodiment, the rotary block is formed with a radially extending rectangular blind hole 31 as a cavity. The top wall of the cavity is formed with a fixing member through hole 33. A fixing member 4 having a ring section 41 is inlaid in the cavity. The rectangular hole 31 has a width 31a along the axis of the rotary block. The width 31a is approximately equal to the width of the ring section 41. The rotary block 3 is further formed with a radially extending through hole 32 intersecting and communicating with the rectangular hole 31. An adjustment rod 5 is fitted through the through hole 32. The diameter of the through hole 32 is equal to the diameter of the adjustment rod 5.

The ring section 41 of the fixing member is formed with a through hole 411. A threaded pillar 42 extending from outer circumference of the ring section is fitted through the fixing member through hole 33 of the cavity. An outer end of the threaded pillar 42 passes through the arced rails 23a, 23b and a pad member 43 bridged between the arced rails. A rotary member 44 is screwed on the threaded pillar to press the pad member against the arced rails 23a, 23b so as to firmly tighten the adjustment rod 5 on the elbow 2.

One end of the adjustment rod 5 extends through the cut 22 of the elbow 2 and is fitted through the through hole 32 of the rotary block and the through hole 411 of the fixing member.

When adjusting the angle and extending length of the adjustment rod 5, a user only needs to unscrew the rotary member 44, permitting the rotary block 3 to be freely rotated. At this time, the angle of the adjustment rod can be changed. Also, the adjustment rod 5 is not tightened so that the adjustment rod can be freely drawn out from the elbow or retracted back into the elbow. Accordingly, the distance between the musical instrument at rear end of the adjustment rod and the performer can be changed.

Thereafter, the rotary member is again screwed and tightened to press the pad member against the arced rails. At this time, the ring section of the fixing member applies a pulling force onto the adjustment rod 5 to press the adjustment rod against inner circumference of the through hole 411. Therefore, the adjustment rod is tightened and fixed. At the same time, the rotary block is fixed.

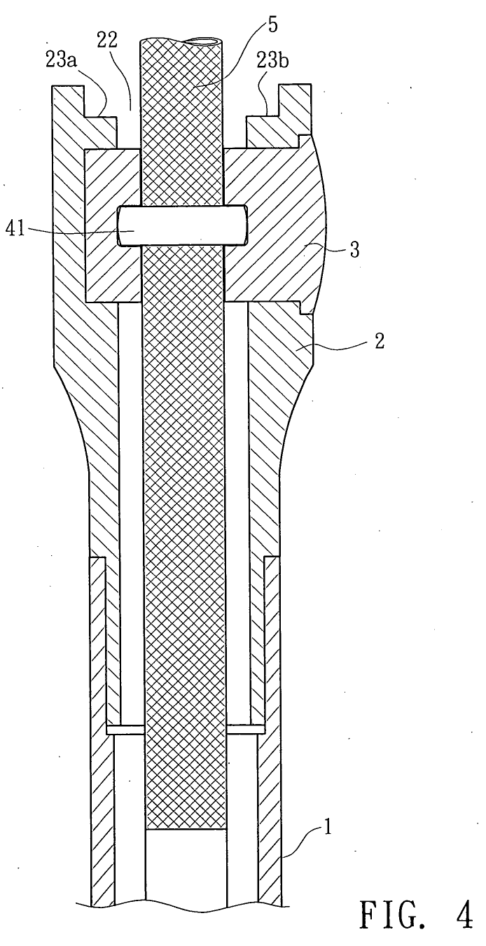

FIG. 4 shows that the adjustment rod 5 is inserted through the stem hole 24 of the bottom of the cut 22 into the upright stem 1. Therefore, the musical instrument stand can be telescoped without occupying much room.

It can be known from the above embodiment that the present invention is characterized in that only by means of unscrewing the rotary member, both the angle and the extending length of the adjustment rod can be adjusted. Therefore, the adjustment is simplified. Moreover, the ring section of the fixing member can apply a greater tightening force onto the adjustment rod for more firmly fix the same.

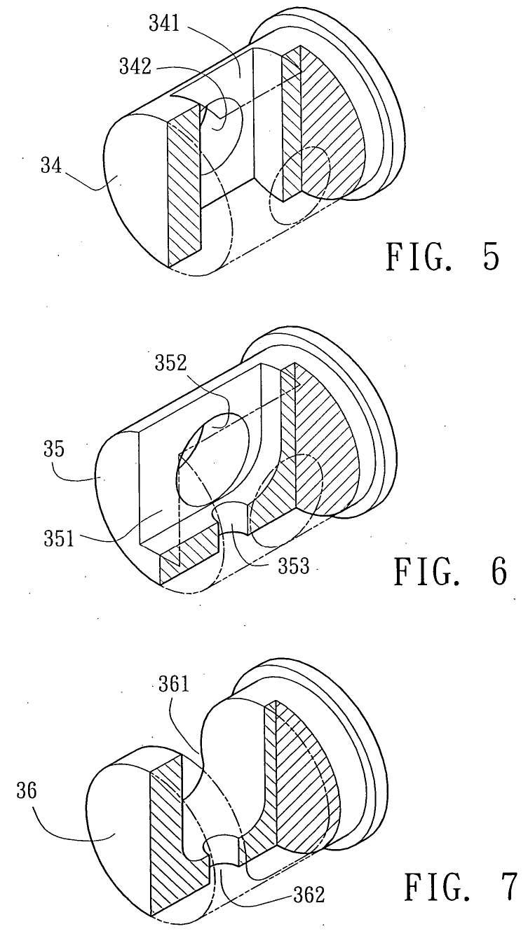

FIG. 5 shows a second embodiment of the present invention, in which the rotary block 34 is formed with a radially extending rectangular through hole 341 as a cavity. A fixing member having a ring section is inlaid in the cavity. The rectangular hole 341 has a width along the axis of the rotary block. The width is approximately equal to the width of the ring section of the fixing member. The rotary block 34 is further formed with a radially extending through hole 342 intersecting and communicating with the rectangular hole 341. An adjustment rod is fitted through the through hole.

FIG. 6 shows a third embodiment of the present invention, in which the rotary block 35 is formed with a cut 351 axially extending from rear end of the rotary block toward front end of the rotary block as a cavity. The bottom of the cut is formed with a fixing member through hole 353. The rotary block is further formed with a radially extending through hole 352 intersecting and communicating with the cut 351. An adjustment rod is fitted through the through hole.

FIG. 7 shows a fourth embodiment of the present invention, in which the rotary block 36 is formed with a radially extending cut 361 intersecting the axis of the rotary block. The bottom of the cut 361 is formed with a fixing member through hole 362. The threaded pillar of the fixing member is fitted through the fixing member through hole 362 with the ring section positioned in the cut 361.

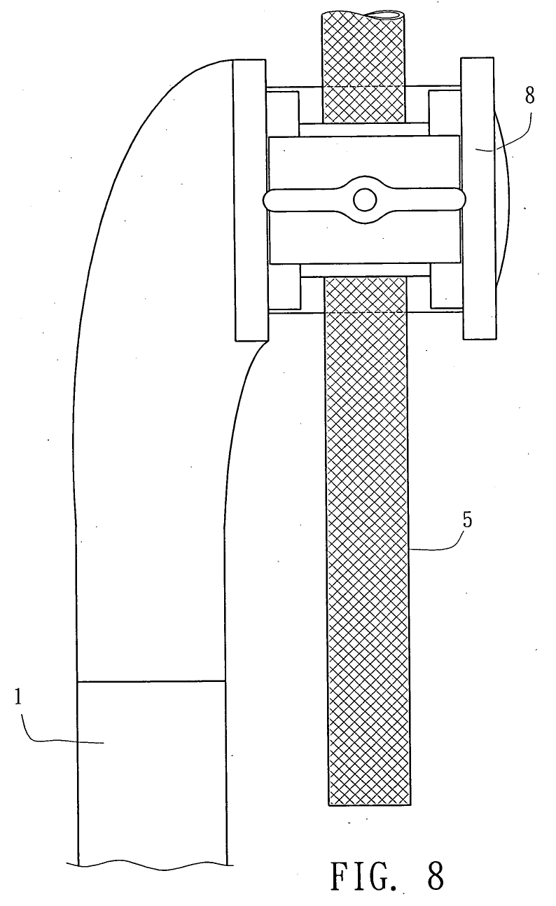

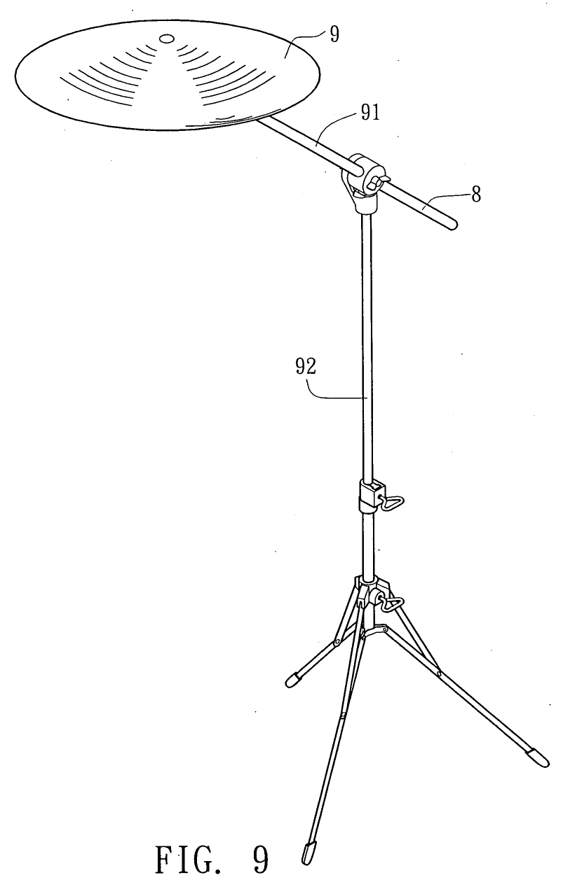

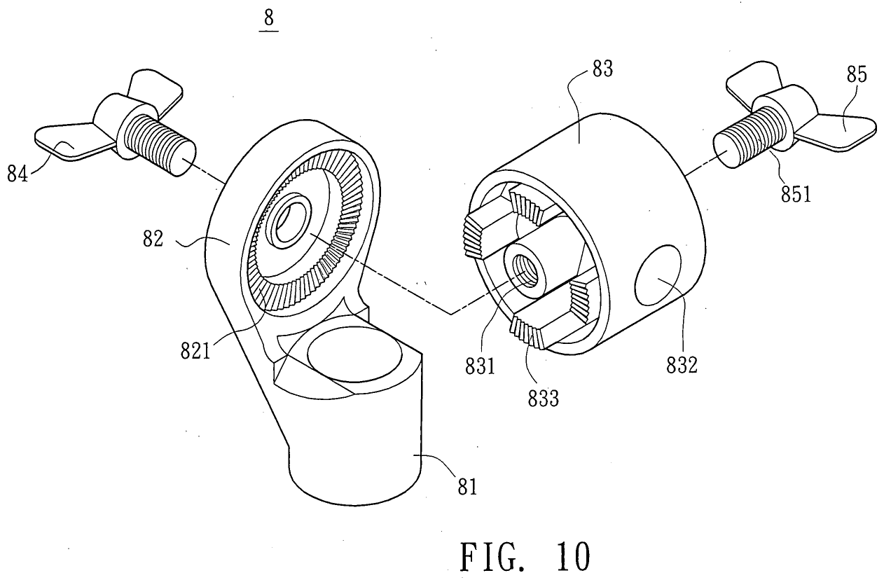

FIG. 8 shows a fifth embodiment of the present invention, in which the upright stem 1 is connected with a lateral side of the elbow 7. Accordingly, the adjustment rod 5 can be positioned in parallel to the upright stem 1. Therefore, the upright stem 1 will not hinder the adjustment rod 5 from being adjusted in inclination angle.

The above embodiments are only used to illustrate the present invention, not intended to limit the scope thereof. Many modifications of the above embodiments can be made without departing from the spirit of the present invention.

What is claimed is:

1. An elbow structure of a musical instrument stand, comprising an elbow connected with an upper end of an upright stem of the stand, the elbow being formed with a transverse circular hole through which a rotary block is fitted, said elbow being characterized in that the elbow is formed with a cut intersecting the circular hole, the cut radially extending within a predetermined arc range of the circular hole, two sides of the cut being respectively formed with two arced rails concentric with the circular hole, the rotary block being formed with a radially extending cavity, a top wall of the cavity being formed with a fixing member through hole, the rotary block being further formed with a through section communicating with the cavity, a fixing member having a ring section being inlaid in the cavity, an adjustment rod being fitted through the through section, the ring section of the fixing member being formed with a through hole, a threaded pillar extending from outer circumference of the ring section being fitted through the fixing member through hole of the cavity, an outer end of the threaded pillar passing through the arced rails and a pad member bridged between the arced rails, a rotary member being screwed on the threaded pillar to press the pad member against the arced rails, one end of the adjustment rod extending through the cut of the elbow and being fitted through the through hole of the rotary block and the through hole of the fixing member.

2. The elbow structure of the musical instrument stand as claimed in claim 1, wherein the cavity is a rectangular hole, the rectangular hole having a width along the axis of the rotary block, the width of the rectangular hole being approximately equal to a width of the ring section of the fixing member, the through section of the rotary block being a through hole communicating with the cavity, the diameter of the through hole of the rotary block being equal to the diameter of the adjustment rod.

3. The elbow structure of the musical instrument stand as claimed in claim 1, wherein the cavity is a cut axially extending from rear end of the rotary block toward front end of the rotary block, the through section of the rotary block being a through hole communicating with the cavity, the diameter of the through hole of the rotary block being equal to the diameter of the adjustment rod.

4. The elbow structure of the musical instrument stand as claimed in claim 1, wherein the rotary block is formed with a radially extending cut intersecting the axis of the rotary block, an interior of the cut being defined as the cavity, two opposite ends of the cut being defined as the through section.

5. The elbow structure of the musical instrument stand as claimed in claim 1, wherein the bottom of the cut of the elbow is formed with a stem hole axially communicating with an interior of the upright stem connected with the elbow.

6. The elbow structure of the musical instrument stand as claimed in claim 1, wherein the upright stem is connected with a lateral side of the elbow.
